(19) United States
(12) Patent Application Publication
(10) Pub. No.: US 2009/0294049 A1
(43) Pub. Date: Dec. 3, 2009

(54) Biodegradable Adhesive Hydrogels

(75) Inventors: Kishore Udipi, Santa Rosa, CA (US); Ya Guo, Rohnert Park, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/131,591

(22) Filed: Jun. 2, 2008

(51) Int. Cl. B32B 37/00; A61F 2/02

(52) U.S. Cl. 156/275.5; 623/23.72

(57) ABSTRACT

Disclosed are methods for forming adhesive hydrogels on tissue surfaces comprising applying to a first tissue surface at a treatment site a first mixture comprising at least one photosensitive molecule and at least one initiator, exposing the first tissue surface to light having a wavelength sufficient to activate the photosensitive molecule to form an activated tissue surface, applying to the activated tissue surface or to a second surface a second mixture comprising at least one macromer, at least one cross-linking agent, and at least one initiator and contacting the first tissue surface with the second surface thus adhering the first tissue surface to the second surface through macromer cross-linking and resulting adhesive hydrogel formation.

### FIELD OF THE INVENTION

The present invention relates to methods for forming biodegradable adhesive hydrogels on tissue surfaces.

### BACKGROUND OF THE INVENTION

Polymers often contain matrices within their macrostructure networks. One type of polymeric matrix is a hydrogel, which can be defined as a water-containing polymeric network. Hydrogels have been beneficially used in medical settings for, for example, bioactive agent delivery, prevention of post-surgical adhesions, tissue repair, etc.

Although there are a variety of methods for producing hydrogels, when these networks are intended to be created in the presence of viable tissue, and/or to contain a bioactive agent, the number of acceptable methods for producing them becomes limited. For example, one method to produce a hydrogel includes solvent casting of hydrophilic polymers. Solvent casting, however, typically involves the use of organic solvents and/or high temperatures which can be detrimental to the activity of bioactive agents and can complicate production methods. Solvent casting of polymers out of solution also results in the formation of non-cross-linked matrices. Non-cross-linked matrices have less structure than cross-linked matrices and, as a result, it can be more difficult to control the release of bioactive agents from such matrices.

U.S. Pat. No. 5,410,016 (Hubbell, et al.) and U.S. Pat. No. 5,529,914 (Hubbell, et al.) relate to the preparation of hydrogels from biodegradable and biostable polymerizable macromers. The hydrogels are prepared from these polymerizable macromers by the use of soluble, low molecular weight initiators. U.S. Pat. No. 5,232,984 (Hubbell, et al.), U.S. Pat. No. 5,380,536 (Hubbell, et al.), U.S. Pat. No. 5,573,934 (Hubbell, et al.), U.S. Pat. No. 5,612,050 (Rowe, et al.), U.S. Pat. No. 5,837,747 (Soon-Shiong, et al.), U.S. Pat. No. 5,846,530 (Soon-Shiong, et al.), and U.S. Pat. No. 5,858,746 (Hubbell, et al.) also describe various methods of forming hydrogels.

Hydrogels formed using such methods, however, often have limited adhesion to tissue. Therefore, additional methods of forming tissue-adhesive hydrogels are needed.

### SUMMARY OF THE INVENTION

The present disclosure provides a method for the in situ synthesis of biodegradable adhesive hydrogels with improved adhesive properties for paving tissue surfaces. In one embodiment, the method of formation is polymerization using photosensitive molecules that initiate a reaction between an amino alcohol initiator, a cross-linking agent, and cross-linkable macromers.

In one embodiment, a method is provided for adhering a first surface to a second surface with an adhesive hydrogel wherein at least one of the first surface and the second surface is a tissue surface, the method comprising: applying to the first surface a first mixture comprising at least one photosensitive molecule and at least one initiator; applying to the second surface a second mixture comprising at least one macromer, at least one cross-linking agent, and at least one initiator; contacting the first surface with the second surface after application of the first and second mixtures; and exposing the contacted surfaces to light having a wavelength sufficient to activate the photosensitive molecule, forming an adhesive hydrogel and adhering the first surface to the second surface.

In one embodiment, a method for forming adhesive hydrogels on tissue surfaces is provided comprising: applying to a first tissue surface at a treatment site a first mixture comprising at least one photosensitive molecule and at least one initiator; applying to the second surface a second mixture comprising at least one macromer, at least one cross-linking agent, and at least one initiator; and contacting the first tissue surface with the second surface after application of the first and second mixtures; and exposing the contacted first tissue surface to light having a wavelength sufficient to activate the photosensitive molecule thus adhering the first tissue surface to the second surface through macromer cross-linking and resulting adhesive hydrogel formation.

In another embodiment, the first mixture further comprises a cross-linking agent.

In another embodiment, the at least one photosensitive molecule is selected from the group consisting of photosensitive dyes, quinones, hydroquinones, poly alkenes, polyaromatic compounds, ketones, unsaturated ketones, peroxides, halides, and derivatives thereof. In yet another embodiment, the photosensitive dye is selected from the group consisting of Eosin Y, Eosin B, fluorone, erythrosine, fluorecsein, indian yellow, and derivatives thereof.

In one embodiment, the at least one initiator is an amino alcohol selected from the group consisting of methyldiethanolamine, triethanolamine, thiols, and amino alcohols having functional groups comprising C₁ to C₁₂ alkyls, C₃ to C₁₂ alkenyls, C₃ to C₁₂ alkynyls, C₆ to C₁₄ aryls, C₄ to C₁₂ heterocyclic alkyls, C₄ to C₁₂ heterocyclic alkenyls, and C₄ to C₁₂ heterocyclic aryls, and derivatives thereof.

In another embodiment, the at least one macromer is selected from the group consisting of polyethers, acrylates, polyesters, polyamides, polyurethanes, poly vinylpyrrolidinone, and derivatives thereof.

In yet another embodiment, the macromer is formed of monomers selected from the group consisting of polyethylene glycol derivatives, trimethylene carbonate, poly vinylpyrrolidinone, poly vinylpyrrolidinone derivatives, hydrophilic polyamides, polyurethanes, polysulfones, acrylates, and derivatives thereof.

In another embodiment, the at least one cross-linking agent is selected from the group consisting of methyl methacrylate, ethyl methacrylate, 2-vinyl pyrrolidinone, propyl methacrylate, hexyl methacrylate, 2-hydroxyethyl methacrylate, lactide, caprolactone, glycolide, butyrolactone, siloxanes, polyethylene glycol, amide containing monomers, and derivatives thereof.

In one embodiment, the second surface is selected from the group consisting of a second tissue surface, a tissue implant, and a medical device. In another embodiment, the tissue implant is selected from the group consisting of organ transplants, cultured cells, cultured tissue, skin, bone, ligaments, blood vessels, and heart valves. In yet another embodiment, the medical device is selected from the group consisting of joint implants, dental implants, soft tissue cosmetic prostheses, wound dressings, vascular prostheses, and ophthalmic prostheses.

In another embodiment, the adhesive hydrogel forms a treatment form selected from the group consisting of tissue adhesives, surgical adhesion prevention barriers, implantable wound dressings, scaffolds for cellular growth, tissue sealants, wound covering agents, controlled release adhesives, and barriers in preventing postoperative adhesions.

In another embodiment, both the first surface and the second surface are a tissue surface. In another embodiment, one of the first surface and the second surface is a tissue surface and the other of the first surface and the second surface is an implantable medical device or a tissue implant. In another embodiment, the tissue implant is selected from the group consisting of organ transplants, cultured cells, cultured tissue, skin, bone, ligaments, blood vessels, and heart valves. In another embodiment, the medical device is selected from the group consisting of joint implants, dental implants, soft tissue cosmetic prostheses, wound dressings, vascular prostheses, and ophthalmic prostheses.

### DEFINITION OF TERMS

Bioactive Agent: As used herein “bioactive agent” shall include any drug, pharmaceutical compound or molecule having a therapeutic effect in an animal. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms, and liposomes.

Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, other rapamycin hydroxyesters and rapamycin hydroxyl ethers may be used.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein “biodegradable” refers to a polymeric composition that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however, all bioresorbable polymers are considered biodegradable. Biodegradable polymers are capable of being cleaved into biocompatible byproducts through chemical- or enzyme-catalyzed hydrolysis.

Cross-linking Agent: As used herein, “cross-linking agent” refers to a monomer that, when polymerized, covalently bonds one polymer chain to another.

Hydrogel: As used herein “hydrogel” refers to a water-containing polymer network.

Initiator: As used herein “initiator” refers to a molecule that initiates a polymerization reaction such as, but not limited to, an amino alcohol.

Macromer: As used herein “macromer” refers to a macromolecule, in particular a polymer, that can be further polymerized or cross-linked.

Photosensitive Molecule: As used herein “photosensitive molecule” refers to a molecule that becomes more reactive when exposed to light (photons).

### DETAILED DESCRIPTION OF THE INVENTION

A method is described herein of forming adhesive hydrogels on a surface comprising initiating the polymerization process on a tissue surface by contacting the tissue surface with a first mixture comprising an initiator, a photosensitive molecule and optionally a cross-linking agent, exposing the tissue surface to a mixture solution comprising at least one macromer, a cross-linking agent and an initiator and exposing the tissue surface to a light, photo-cross-linking the macromers such that a hydrogel with improved adhesive properties is formed on the surface.

The adhesive hydrogels are biocompatible and biodegradable. Macromers suitable for forming the adhesive hydrogels are formed of monomers including, but not limited to, polyethylene glycol derivatives, trimethylene carbonate, poly vinylpyrrolidinone, poly vinylpyrrolidinone derivatives, hydrophilic polyamides, polyurethanes, polysulfones, acrylates, and derivatives thereof.

The adhesive hydrogels are useful for biomedical applications such as, but not limited to, tissue adhesives, surgical adhesion prevention barriers, implantable wound dressings, scaffolds for cellular growth, tissue sealants, wound covering agents, controlled release adhesives, and barriers in preventing postoperative adhesions.

Furthermore, the adhesive hydrogels can be used, for example, to provide adhesion between two tissue surfaces or between a tissue surface and the surface of a medical device. When the adhesive hydrogels provide adhesion between a tissue surface and the surface of a medical device, the medical device can be provided at the treatment site coated with at least one component of the hydrogel forming mixture. The hydrogel forming mixtures can be applied to the treatment site and/or the medical device either before, during, or after implantation of the medical device at the treatment site.

The polymerization process for the hydrogels is conducted in situ and comprises applying a first mixture comprising at least one photosensitive molecule, at least one initiator, and optionally at least one cross-linking agent to a tissue surface. A second mixture comprising at least one initiator, at least one cross-linking agent, and at least one polymerizable macromer is then applied to the tissue surface. The tissues, or surfaces to be adhered, are then contacted with each other. The tissue is then exposed to light of an appropriate wavelength to excite the photosensitive molecule. Free radicals formed during the activation interact with the initiator and the cross-linking agent which, in turn, causes the polymerization and cross-linking of the macromer to form an adhesive hydrogel that adheres the two surfaces together.

Photosensitive molecules suitable for forming the adhesive hydrogels include, but are not limited to, photosensitive dyes, quinones, hydroquinones, poly alkenes, polyaromatic compounds, ketones, unsaturated ketones, peroxides, halides, Eosin Y, Eosin B, flourone, erythrosine, flourecsein, indian yellow, derivatives thereof, and/or combinations thereof. In one embodiment, the photosensitive molecule is Eosin Y. In another embodiment, the photosensitive molecule is Eosin B.

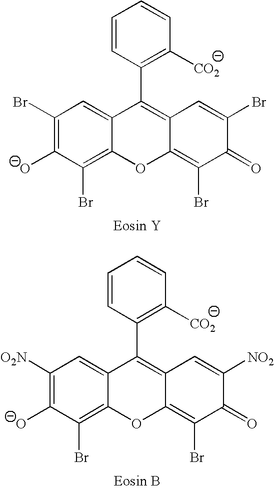

In another embodiment, the photosensitive molecule is fluorone.

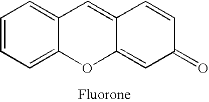

In another embodiment, the photosensitive molecule is erythrosine.

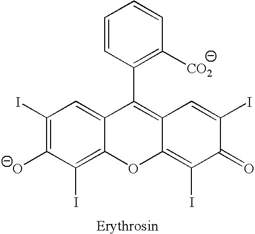

In another embodiment, the photosensitive molecule is fluorescein.

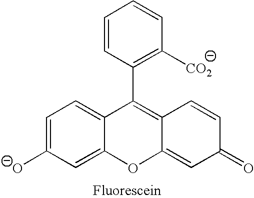

In another embodiment, the photosensitive molecule is indian yellow.

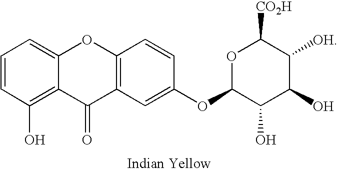

Each of the photosensitive molecules disclosed herein requires exposure to light of an appropriate excitation wavelength to activate the molecule. Each photosensitive molecule may have a different or similar excitation wavelength. For example, and not intended as a limitation, Eosin B is activated by light having wavelengths of 511-520 nm. In a further example also not intended as a limitation, Eosin Y is activated by light having a wavelength of approximately 490 nm. Excitation wavelengths of photosensitive molecules are well known to persons of ordinary skill in the art. Exemplary photosensitive molecules and the excitation wavelengths are described in U.S. Pat. No. 6,602,975 issued to Hubbell et al. which is incorporated by reference for all it contains regarding photosensitive molecules.

Initiator molecules suitable for forming the adhesive hydrogels include amino alcohols such as, but not limited to, methyldiethanolamine, triethanolamine, thiols, and amino alcohols having functional groups comprising C₁ to C₁₂ alkyls, C₃ to C₁₂ alkenyls, C₃ to C₁₂ alkynyls, C₆ to C₁₄ aryls, C₄ to C₁₂ heterocyclic alkyls, C₄ to C₁₂ heterocyclic alkenyls, and C₄ to C₁₂ heterocyclic aryls, derivatives thereof, and/or combinations thereof.

Cross-linking agents suitable for forming the adhesive hydrogels include, but are not limited to, methyl methacrylate, ethyl methacrylate, 2-vinyl pyrrolidinone, propyl methacrylate, hexyl methacrylate, 2-hydroxyethyl methacrylate, lactide, caprolactone, glycolide, butyrolactone, siloxanes, polyethylene glycol, amide containing monomers, and derivatives thereof.

The in situ formation of hydrogels on tissue surfaces is further disclosed. More specifically, a method to cross-link macromers with polymers or to simply polymerize the macromers to improve the adhesive properties of the hydrogel is provided. In one embodiment, the photosensitive molecule is evenly spread on the tissue to be treated along with an initiator molecule and optionally a cross-linking agent in a first mixture. The tissue is then coated with macromeric components of the hydrogel and an initiator molecule such as, but not limited to, methyldiethanolamine or triethanolamine, and a cross-linking agent. The photosensitive molecule, upon activation by light, forms a free radical and abstracts a proton from the initiator molecule which in turn attacks the cross-linking agent thereby cross-linking the macromer. The adhesive hydrogel formed in this manner provides adhesion between two tissue surfaces or between a tissue surface and a medical device or a tissue implant.

In another embodiment, the adhesive hydrogel further comprises at least one bioactive agent. The bioactive agent can be applied to the treatment site in either the first mixture or the second mixture, or both mixtures.

Furthermore, the adhesive hydrogel can also have cells or tissues deposed therein.

As described herein, the adhesive hydrogel can thus be used to fill the spaces between a tissue implant or medical device (itself either tissue-based or non-tissue based) and adjacent tissue. Non-limiting exemplary tissue implants include both those obtained as transplants (e.g., autografts, allografts, or xenografts) and those provided by tissue engineering. Such tissue implants do not typically conform well to adjacent native tissue however, thus leaving spaces into which undesirable fluids and cells can accumulate and produce adverse tissue responses. For example, when cultured cartilage is implanted into cartilage defects, synovial fluid and macrophages can enter the unfilled space and lead to fibrous tissue formation, which prevents integration of the implanted cartilage with the native cartilage. Other cultured tissues that are implanted into tissue defects, and that would benefit from the present macromer system applied as a grout include, but are not limited to, skin, bone, ligaments, blood vessels, and heart valves.

Exemplary medical devices include those in which tissue integration is desired, such as those that provide a sufficiently porous surface or can have a porous surface provided thereon including, but not limited to, joint implants (e.g., for hip or knee reconstruction), dental implants, soft tissue cosmetic prostheses (e.g., breast implants), wound dressings, vascular prostheses (e.g., vascular grafts and stents), and ophthalmic prostheses (e.g., intracorneal lenses). The adhesive hydrogel can be used in any suitable manner (e.g., to coat and/or fill voids within or upon the surface of the medical device).

The adhesive hydrogel systems can be applied to a tissue site and/or medical device in any suitable manner, including by spraying, dipping, injecting or brushing the first mixture and/or the second mixture on a substrate surface prior to cross-linking.

### EXAMPLE 1

### Synthesis of an Exemplary Macromer

Polyethyleneglycol (PEG), PEG3400 (30 g, Mn340ODa, Sigma Aldrich, St. Louis, Mo.) and trimethylene carbonate(TMC) (2.7 g) were added into a three neck flask and purged with nitrogen for at least 20 min. Tin octoate (7.15 g) was added as catalyst. The flask was heated to 90° C. using an oil bath and the reaction was allowed to proceed for 12 hr. The PEG/TMC polymer was dissolved into tetrahydrofuran (THF) and precipitated into ether.

The PEG/TMC polymer was acrylated as follows. To form PEG/TMC diacrylate, 10 g of PEGTMC was dissolved into 150 mL of anhydrous chloromethane and purged with nitrogen. 0.59 g of Acryloyl chloride and 0.66 g of triethylamine were added dropwise into the solution. The mixture was stirred and refluxed at 40-50° C. overnight under nitrogen. After reaction, the solution was filtered and precipitated into ether. The macromer created has the structure of formula 1.

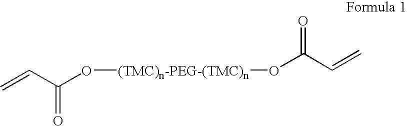

### EXAMPLE 2

### Forming an Adhesive Hydrogel on a Tissue

An appropriate vessel wall in need of therapy is chosen. Eosin Y and methyldiethanolamine are applied on the tissue in need thereof. Then, macromers of formula 1, polyvinylpyrrolidinone, and methyldiethanolamine are further added to the tissue surface. Light at 532 nm, to activate eosin Y, is applied to the tissue and an adhesive hydrogel is formed.

### EXAMPLE 3

### Connecting Two Surfaces Using an Adhesive Hydrogel

An appropriate vessel wall in need of therapy is chosen. Eosin Y and methyldiethanolamine are applied on the tissue in need thereof. Then, macromers of formula 1, polyvinylpyrrolidinone, and methyldiethanolamine are applied to the surface of a stent. The stent is implanted into the vessel in need thereof. Then, light at 532 nm, to activate eosin Y, is applied to the tissue and an adhesive hydrogel is formed thereby adhering the stent to the tissue surface.

Unless otherwise indicated, all numbers expressing quantities of ingredients, properties such as molecular weight, reaction conditions, and so forth used in this specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on the described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for adhering a first surface to a second surface with an adhesive hydrogel wherein at least one of said first surface and said second surface is a tissue surface, said method comprising:
a) applying to said first surface a first mixture comprising at least one photosensitive molecule and at least one initiator;
b) applying to said second surface a second mixture comprising at least one macromer, at least one cross-linking agent, and at least one initiator;
c) contacting said first surface with said second surface after application of said first mixture and said second mixture; and
d) exposing said contacted surfaces to light having a wavelength sufficient to activate said photosensitive molecule, thereby forming an adhesive hydrogel and adhering said first surface to said second surface.

2. The method according to claim 1 wherein both of said first surface and said second surface are a tissue surface.

3. The method according to claim 1 wherein one of said first surface and said second surface is a tissue surface and the other of said first surface and said second surface is an implantable medical device or a tissue implant.

4. The method according to claim 3 wherein said tissue implant is selected from the group consisting of organ transplants, cultured cells, cultured tissue, skin, bone, ligaments, blood vessels, and heart valves.

5. The method according to claim 3 wherein said medical device is selected from the group consisting of joint implants, dental implants, soft tissue cosmetic prostheses, wound dressings, vascular prostheses, and ophthalmic prostheses.

6. The method according to claim 1 wherein said first mixture further comprises a cross-linking agent.

7. The method according to claim 1 wherein said at least one photosensitive molecule is selected from the group consisting of photosensitive dyes, quinones, hydroquinones, poly alkenes, polyaromatic compounds, ketones, unsaturated ketones, peroxides, halides, and derivatives thereof.

8. The method according to claim 7 wherein said photosensitive dye is selected from the group consisting of Eosin Y, Eosin B, fluorone, erythrosine, fluorecsein, indian yellow, and derivatives thereof.

9. The method according to claim 1 wherein said at least one initiator is an amino alcohol selected from the group consisting of methyldiethanolamine, triethanolamine, thiols, and amino alcohols having functional groups comprising C₁ to C₁₂ alkyls, C₃ to C₁₂ alkenyls, C₃ to C₁₂ alkynyls, C₆ to C₁₄ aryls, C₄ to C₁₂ heterocyclic alkyls, C₄ to C₁₂ heterocyclic alkenyls, C₄ to C₁₂ heterocyclic aryls, and derivatives thereof.

10. The method according to claim 1 wherein said at least one macromer is selected from the group consisting of polyethers, acrylates, polyesters, polyamides, polyurethanes, poly vinylpyrrolidinone, and derivatives thereof.

11. The method according to claim 10 wherein said at least one macromer is formed of monomers selected from the group consisting of polyethylene glycol derivatives, poly vinylpyrrolidinone, trimethylene carbonate, poly vinylpyrrolidinone derivatives, hydrophilic polyamides, polyurethanes, polysulfones, acrylates, and derivatives thereof.

12. The method according to claim 1 wherein said at least one cross-linking agent is selected from the group consisting of methyl methacrylate, ethyl methacrylate, 2-vinyl pyrrolidinone, propyl methacrylate, hexyl methacrylate, 2-hydroxyethyl methacrylate, lactide, caprolactone, glycolide, butyrolactone, siloxanes, polyethylene glycol, amide containing monomers, and derivatives thereof.

13. A method for forming adhesive hydrogels on tissue surfaces comprising:
a) applying to a first tissue surface at a treatment site a first mixture comprising at least one photosensitive molecule and at least one initiator;
b) applying to said first tissue surface a second mixture comprising at least one macromer, at least one cross-linking agent, and at least one initiator; and
c) contacting said first tissue surface with a second surface after application of said first mixture and said second mixture; and
d) exposing said contacted first tissue surface to light having a wavelength sufficient to activate said photosensitive molecule thus adhering said first tissue surface to said second surface through macromer cross-linking and resulting adhesive hydrogel formation.

14. The method according to claim 13 wherein said first mixture further comprises a cross-linking agent.

15. The method according to claim 13 wherein said at least one photosensitive molecule is selected from the group consisting of photosensitive dyes, quinones, hydroquinones, poly alkenes, polyaromatic compounds, ketones, unsaturated ketones, peroxides, halides, and derivatives thereof.

16. The method according to claim 15 wherein said photosensitive dye is selected from the group consisting of Eosin Y, Eosin B, fluorone, erythrosine, fluorecsein, indian yellow, and derivatives thereof.

17. The method according to claim 13 wherein said at least one initiator is an amino alcohol selected from the group consisting of methyldiethanolamine, triethanolamine, thiols, and amino alcohols having functional groups comprising C₁ to C₁₂ alkyls, C₃ to C₁₂ alkenyls, C₃ to C₁₂ alkynyls, C₆ to C₁₄ aryls, C₄ to C₁₂ heterocyclic alkyls, C₄ to C₁₂ heterocyclic alkenyls, C₄ to C₁₂ heterocyclic aryls, and derivatives thereof.

18. The method according to claim 13 wherein said at least one macromer is selected from the group consisting of polyethers, acrylates, polyesters, polyamides, polyurethanes, poly vinylpyrrolidinone, and derivatives thereof.

19. The method according to claim 18 wherein said macromer is formed of at least one monomer selected from the group consisting of polyethylene glycol derivatives, poly vinylpyrrolidinone, trimethylene carbonate, poly vinylpyrrolidinone derivatives, hydrophilic polyamides, polyurethanes, polysulfones, acrylates, and derivatives thereof.

20. The method according to claim 13 wherein said at least one cross-linking agent is selected from the group consisting of methyl methacrylate, ethyl methacrylate, 2-vinyl pyrrolidinone, propyl methacrylate, hexyl methacrylate, 2-hydroxyethyl methacrylate, lactide, caprolactone, glycolide, butyrolactone, siloxanes, polyethylene glycol, amide containing monomers, and derivatives thereof..

21. The method according to claim 13 wherein said second surface is selected from the group consisting of a second tissue surface, a tissue implant, and a medical device.

22. The method according to claim 21 wherein said tissue implant is selected from the group consisting of organ transplants, cultured cells, cultured tissue, skin, bone, ligaments, blood vessels, and heart valves.

23. The method according to claim 21 wherein said medical device is selected from the group consisting of joint implants, dental implants, soft tissue cosmetic prostheses, wound dressings, vascular prostheses, and ophthalmic prostheses.

24. The method according to claim 13 wherein said adhesive hydrogel forms a treatment form selected from the group consisting of tissue adhesives, surgical adhesion prevention barriers, implantable wound dressings, scaffolds for cellular growth, tissue sealants, wound covering agents, controlled release adhesives, and barriers in preventing postoperative adhesions.
